(19) United States
(12) Patent Application Publication
(10) Pub. No.: US 2012/0244568 A1
(43) Pub. Date: Sep. 27, 2012

(54) LABEL-FREE RIGID CELL ASSAY METHOD

(76) Inventors: Fang Lai, Painted Post, NY (US); Guangshan Li, Painted Post, NY (US); Anping Liu, Horseheads, NY (US); Jinlin Peng, Painted Post, NY (US)

(21) Appl. No.: 13/357,969

(22) Filed: Jan. 25, 2012

Related U.S. Application Data
(60) Provisional application No. 61/466,264, filed on Mar. 22, 2011.

(51) Int. Cl. C12Q 1/02

(52) U.S. Cl. 435/29

(57) ABSTRACT

A label-free cell assay method including:
culturing rigid cells in a buffer, the rigid cells having a diameter of about 3 to about 7 micrometers;
depositing the rigid cells on the surface of a sensor system; and
detecting the rigid cells with the sensor when exposed to two or more wavelengths of light having two or more penetration depths, as defined herein.

Also disclosed are assay methods for contacting the rigid cells with a substance and determining the response of the contacted rigid cells to the substance, such as a drug candidate or modulator compound, as defined herein.

This application claims the benefit of priority under 35 U.S.C. §119 of U.S. Provisional Application Ser. No. 61/466,264, filed on Mar. 22, 2011, the content of which is relied upon and incorporated herein by reference in its entirety.

### BACKGROUND

The disclosure relates generally to biosensing, and to a label-free rigid cell assay method.

### SUMMARY

The disclosure provides methods for real-time SPR biosensing using multiple wavelengths, and more specifically the disclosure provides a method for label-free rigid cell detection and assay using multiple depth DRCA™ sensing system.

### BRIEF DESCRIPTION OF THE DRAWING(S)

In embodiments of the disclosure:

FIG. 1 is a schematic which illustrates the penetration effect on rigid cells having a generally spherical geometry and a diameter of about 3 to about 7 microns.

FIG. 2 charts modeled signal amplitude responses for selected illuminating laser wavelengths on a simulated rigid spherical body.

FIG. 3 charts yeast cell response curves resulting from the use of three different illuminating wavelengths.

FIG. 4 illustrates the effect of various substances on yeast cell growth measured at three different illuminating wavelengths.

FIG. 5 illustrates the time course of the effect of amphotericin (Amph) on yeast cell growth.

FIG. 6 charts data collected after about 16 hours of a yeast cell assay using the different illuminating wavelengths.

FIG. 7 charts absorbance readings from a conventional spectrophotometer for yeast cells contacted with and without amphotericin.

FIG. 8 charts cell counts obtained with a conventional hemocytometer.

FIG. 9 shows a typical logarithmic growth curve for yeast cells with respect to time and potential culture harvest points or regions.

FIG. 10 shows response units for yeast cells and an apparent dose-dependent effect on the yeast cells for each of the different illuminating wavelengths.

FIG. 11 shows an exemplary schematic of a multi-wavelength SPR sensing system using a sensor chip.

### DETAILED DESCRIPTION

Various embodiments of the disclosure will be described in detail with reference to drawings, if any. Reference to various embodiments does not limit the scope of the invention, which is limited only by the scope of the attached claims. Additionally, any examples set forth in the specification are not limiting and merely set forth some of the many possible embodiments for the claimed invention.

### Definitions

“Substance” or like term refers to any agent or material that can influence a cell's biology, including, for example, a molecule, biomolecule, a compound, an oligomer, a polymer, a modulator, an effector, a nutrient, a toxin, an inhibitor, a gas, radiation, electricity, and like entity, or combinations thereof.

“Modulator” or like term refers to anything that can affect cell signaling, including a compound, a biomolecule, or other agency that can affect cell signaling. Modulator can refer to a molecule that binds to a regulatory site during allosteric modulation and allosterically modulates the shape of the protein, or more generally, any molecule that can trigger another molecular response.

“Effector” or like term refers to a molecule, including the spectrum of small molecules and encompassing any regulatory molecule, including proteins, that can bind to a protein and alter the activity of that protein, or biological cells that interact with other cells.

“Compound” refers to a discrete chemical entity, such as a small molecule of molecular weight of from 20 to about 500, or a molecule of greater molecular weight of from 500 to about 10,000,000, including oligomers and polymers, and can be a gas, a liquid, a solid, or a combination thereof.

“Bio-molecule” refers to a compound or like entity of biological origin or a synthetic equivalent or synthetic modification of a compound of biological origin, such as DNA, RNA, peptide, protein, antibody, conjugates thereof, or like entity.

“Include,” “includes,” or like terms mean encompassing but not limited to, that is, inclusive and not exclusive.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example: through typical measuring and handling procedures used for making compounds, compositions, composites, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. The claims appended hereto include equivalents of these “about” quantities.

“Consisting essentially of” in embodiments refers, for example, to a variable depth sensor and sensor system, and to a method of making or using the variable depth sensor and sensor system, and articles, devices, or any apparatus of the disclosure, and can include the components or steps listed in the claim, plus other components or steps that do not materially affect the basic and novel properties of the articles, apparatus, or methods of making and use of the disclosure, such as particular additives or ingredients, a particular agent, a particular surface modifier or condition, or like structure, material, or process variable selected. Items that may materially affect the basic properties of the components or steps of the disclosure or that may impart undesirable characteristics to aspects of the disclosure include, for example, unintentional or inadvertent substance deterioration, or like functional disruption, changes to, or contamination of the substance's molecular structure or characteristics by chemical or physical means, which changes could confound the cellular or nuclear characteristics of the assay.

The indefinite article “a” or “an” and its corresponding definite article “the” as used herein means at least one, or one or more, unless specified otherwise.

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, and like abbreviations).

Specific and preferred values disclosed for components, ingredients, additives, reactants, reagents, polymers, oligomers, monomers, times, temperatures, and like aspects, and ranges thereof, are for illustration only; they do not exclude other defined values or other values within defined ranges. The compositions and methods of the disclosure include those having any value or any combination of the values, specific values, more specific values, and preferred values described herein.

Fungi are a large group of eukaryotic microorganisms including yeasts, molds, and mushrooms. One major difference among fungi, plants, and bacteria is that fungal cells have cell walls that contain chitin. In contrast the cell walls of plants contain cellulose and bacterial walls contain peptidoglycan. Abundant worldwide, most fungi are inconspicuous because of the small size of their structures, and their cryptic lifestyles in soil, on dead matter, and as symbionts of plants, animals, or other fungi. Fungi perform an essential role in the decomposition of organic matter and have fundamental roles in nutrient cycling and exchange. They have long been used as a direct source of food, such as mushrooms and truffles, as a leavening agent for bread, and in fermentation of various food products, such as wine, beer, and soy sauce. Since the 1940s, fungi have been used for the production of antibiotics, and, more recently, various enzymes produced by fungi are used industrially and in detergents. Fungi are also used as biological agents to control weeds and pests. Many fungi species produce bioactive compounds called mycotoxins, such as alkaloids and polyketides that are toxic to animals including humans.

Yeasts are unicellular, although some species with yeast forms may become multicellular through the formation of a string of connected budding cells. Yeast size can vary greatly depending on the species, typically measuring 3 to 7 micrometers in diameter. Some yeast species, such as Candida albicans, are opportunistic pathogens and can cause infections in humans. The yeast species Saccharomyces cerevisiae is perhaps the most useful yeast, and has been used in baking and fermenting alcoholic beverages for thousands of years (see J-L Legras, et al., “Bread, beer and wine: Saccharomyces cerevisiae diversity reflects human history,” Molecular Ecology, 16: 2091-2102 (2007)). Recently, yeasts have been used to generate electricity in microbial fuel cells (see “Biofuel cell,” Helsinki University of Technology, 2007), and to produce ethanol for the biofuel industry (see “Towards Sustainable Production and Use of Resources: Assessing Biofuels,” United Nations Environment Program.).

S. cerevisae is also important as a model organism in modern cell biology research, and is one of the most thoroughly researched eukaryotic microorganisms. Researchers have used it to gather information about the biology of the eukaryotic cell and ultimately human biology (see Ostergaard S. et al., “Metabolic engineering of Saccharomyces cerevisiae”, Microbiology and Molecular Biology Reviews, 64 (1): 34-50 (2000)). S. cerevisiae cells are round to ovoid, and about 3 to about 7 micrometers in diameter. It reproduces by a division process known as budding. S. cerevisiae was the first eukaryotic genome that was completely sequenced. The genome is composed of about 12,156,677 base pairs and 6,275 genes, compactly organized on 16 chromosomes. S. cerevisiae has been developed as a model organism because it scores favorably on a number of criteria, such as a single celled organism, easily genetically engineered, shares complex internal cell structure of plants and animals without the high percentage of non-coding DNA, and defined metabolic pathways. For these reasons, many yeast cell assays have been developed to study protein-protein interactions (two yeast hybrid), to evaluate the cytotoxicity and cytostaticity of natural compounds or synthetic compounds (see Poletto, N. P., et al., “Evaluation of Cytotoxic and Cytostatic Effects in Saccharomyces cerevisiae by Poissoner Quantitative Drop test,” Basic Clin Pharmacol Toxicol, 104(1):71-75(2009)), and to identify new chemicals that interact with potential drug targets (Gaido, K. W., et al., Evaluation of Chemicals with Endocrine Modulating Activity in a Yeast-Based Steroid Hormone Receptor Gene Transcription, Toxicology and Applied Pharmacology, 143(1):205-212(1997)). While powerful, the assays for the discovery of interactions between chemicals and targets often require the lysis of cells for measuring the activity, which can be cumbersome.

In recent years, many label-free cell based assays have been developed using optical or electrical sensors, such as Epic® assay and impedance assay. The Epic® assay uses a resonant waveguide grating to sense the reflective or refractive index change(s) as a result of mass change near the sensing surface. The assay is very sensitive in detecting the mass change of mammalian cells and even bacterial cells, see commonly owned and assigned copending application U.S. Ser. No. 12/956,464. However, the Epic® assay has heretofore not been used for yeast cell assays, especially for S. cerevisiae, since this yeast is spherically shaped, nominally about 5 microns in diameter, and has a rigid cell wall. When this yeast sits on a sensor, the yeast cell only touches a small area of the surface, resulting in very little mass increase or decrease within the 150 to 200 nm sensing region. To enable a label-free yeast cell assay, an increased sensing depth is a prerequisite.

Initial failure in detecting yeast cells at the highest seeding density of a serial dilution with the Epic® platform led to a presumption that a penetration depth greater than 200 nm is required for sensing the presence of yeast cells. Referring to FIG. 1, hypothetical or representative rigid cells are substantially round, and create a large void-area and void-volume near the sensing surface. To test the hypothesis, a DRCA system, equipped with three different lasers at wavelengths of 660 nm, 980 nm, and 1480 nm, was deployed. Based on the modeling and simulation with a gold coated glass substrate, these wavelengths translated into sensing depths of about 200 nm (660 nm laser), about 570 nm (980 nm laser), and about 1500 nm (1480 nm laser). With the DRCA system, it was possible to prove that a deep penetration depth is significant for sensing the presence of yeast cells. Furthermore, it was also demonstrated that the disclosed sensing methodology could be used to assay rigid cells having a spherical-shape, oval-shape, or egg-shape, such as budding yeast.

In embodiments, the disclosure provides a method to simultaneously monitor yeast or like cells with multiple depths, making a label-free rigid cell assay possible. The ability to measure non-rigid cell response at a deeper penetration depth with a Depth Resolved Cell Assay (DRCA) system has been previously demonstrated, see commonly owned and assigned copending applications U.S. Ser. No. 12/956,464, and U.S. Ser. No. 12/947,066, both filed Nov. 11, 2009, the disclosures of which are entirely incorporated here by reference. Additional improvements in the DRCA™ system now provide the ability to simultaneously sense multiple depths of a cell. The DRCA™ system can be used to monitor the yeast cell response in real-time. This makes the DRCA™ system useful for, for example, yeast biological study, drug discovery, or both, but also makes the DRCA™ system more versatile for an extended range of cell assays across many more species.

Other background apparatus and operational aspect of the present disclosure are mentioned in commonly owned and assigned copending application U.S. Ser. No. 12/955421, filed Nov 29, 2010, entitled “SYSTEMS AND METHODS FOR MULTI-WAVELENGTH SPR BIOSENSING WITH REDUCED CHROMATIC ABERRATION,” the disclosure of which is entirely incorporated here by reference.

Referring again to FIG. 1, a biosensor system 100 having a sensing surface 110 and a budding yeast cell ensemble 120 that is substantially round or spherical, having a diameter of about 3 to about 7 micrometers, and having a rigid cell wall is shown. When situated on a surface in a substantially monolayer, the yeast cells 120 can have a relatively large void-area on the surface and a relatively large void-volume 130 near the conventional sensing region or within the detection zone 150, that is, extending vertically away from the surface by about 150 to 200 nanometers. The void-area and void-volume 130 initially decreases gradually moving away from the surface, then decreases more rapidly and approaches zero near the equatorial contact points between the yeast cell buds. A conventional Epic® biosensor system can barely detect the presence of yeast cells due to the limited near sensing depth or near detection zone 150 of about 150 nm to about 200 nm. Using a DRCA system equipped with multiple light sources covering a broad range of wavelengths, sensing a yeast cell at increased penetration depths 140, for example, from about 200 to about 1,500 nm, and from about 500 to about 1,500 nm, is enabled. In embodiments, the disclosure demonstrates the use of the DRCA system for directly monitoring yeast cells and the yeast cell response to external stimuli.

In embodiments, the disclosure provides a number of advantages over existing yeast cell or like cell assay methods. The ability to simultaneously monitor a spherical and rigid cell of a species, such as yeast, coccus bacteria, or plant cells, using multiple depth label-free sensors provides significant advantages over conventional methods. For example, in embodiments the disclosed methods provides a methodology that permits one to detect, monitor, or measure a live-yeast cell or a cell response in real time with different penetration depths, which can provide greater information that is also physiologically relevant compared to known methods. The methodology can also be used for directly sensing the effect of substances, such as modulators, or like materials, on yeast cells, or like cells, to provide a significantly simplified assay protocol. The budding yeast is the simplest eukaryotic cell model. Having the ability to assay yeast cells in a label-free manner provides a new way to study rigid cell biology.

In embodiments, the disclosed method enables a label-free rigid cell assay, which was heretofore unavailable or inaccessible using the existing label-free methods.

In embodiments, the disclosed method is applicable to any cell type that is approximately spherical or oval, and relatively rigid, for example, non-flattening or individually non-spreading. The disclosed method significantly broadens the spectrum of label-free cell assays available to a user of a gold chip surface plasmon resonance optical sensor system.

In embodiments, the disclosure provides a label-free cell detection or assay method comprising, for example:

culturing rigid cells in a buffer, the rigid cells having a diameter of about 3 to about 7 micrometers;

depositing the rigid cells on the surface of a gold chip in a surface plasmon resonance optical sensor system; and

detecting the presence of the rigid cells on the sensor surface with the sensor system based on refraction changes when the deposited rigid cells are exposed, for example, sequentially or simultaneously, to two or more wavelengths of light having two or more penetration depths.

In embodiments, the method can further comprise, or independently comprise when in combination with the abovementioned detection method:

contacting the deposited rigid cells with a substance; and

determining the differential response of the contacted rigid cells to the substance compared to rigid cells not contacted by the substance.

Determining the differential response of the rigid cells to the substance can comprise, for example, at least one of:

measuring a baseline response of a buffer;

measuring a baseline response of the buffer and the rigid cells with the two or more wavelengths having two or more penetration depths;

measuring a response of the buffer and the substance contacted rigid cells with the two or more wavelengths having two or more penetration depths; and computing the difference between the measured response of the contacted rigid cells and at least one baseline response.

In embodiments, the two or more wavelengths can be, for example, a plurality of wavelengths, such 2, 3, 4, and up to 20 different wavelengths, including intermediate values and ranges, and the two or more penetration depths can be, for example, a plurality of penetration depths such 2, 3, 4, and up to 10 different penetration depths, including intermediate values and ranges. In embodiments, a single wavelength can be selected by experiment, for example, a single wavelength having the penetration depth that is best matched to the dimensions of the rigid cell specimen to provide the maximum response.

In embodiments, the sensor system can include, for example:

a light source providing at least two incident beams, each beam having at least one wavelength;

first optics providing incident beam shaping and beam focusing;

a sensor chip comprising a transparent substrate having on the first face of the substrate a high refractive index prism for receiving the incident focused beam, and having on the second face of the substrate having a metal layer of from about 30 nm to about 100 nm;

second optics providing reflected or emitted beam collection from the sensor;

a photodetector for receiving the collected beam and detecting the optical signal; and

a data acquisition unit, and the cells are situated on the metal layer of the sensor chip surface.

In embodiments, the rigid cells can have, for example, a spherical or elliptical shape and have an aspect ratio of from about 1:1 to about 1:4, including intermediate values and ranges. The rigid cells can be, for example, substantially non-deformable or only slightly deformable, and generally do not change their aspect ratio by more than, for example, from about 1 to about 25%, from about 2 to about 20%, about 3 to about 15%, and like change percentages, including intermediate values and ranges, during, for example, culturing, detecting, assaying, or a combination thereof.

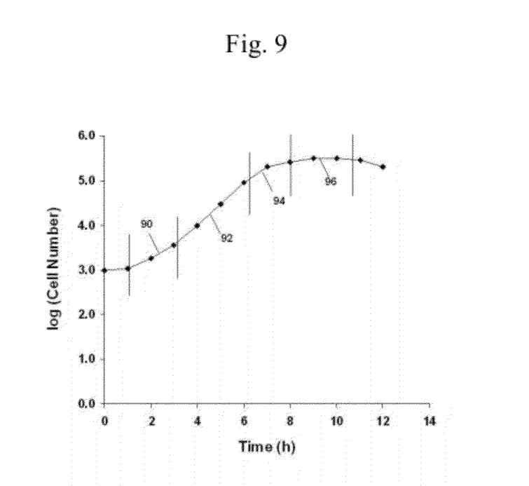

In embodiments, the contacted rigid cells of the substance assay can be collected, for example, at the logarithm growth stage (FIG. 9) prior to contacting with the substance. In embodiments, the logarithm stage can be, for example, from the early (inflection point) stage of the growth curve to the late (plateau) stage of the growth curve. In embodiments, the contacted cultured rigid cells can be, for example, collected at the late logarithm stage and prior to contacting with the substance. In embodiments, the deposited rigid cells can be, for example, collected at the late logarithm stage of culture and prior to contacting.

In embodiments, culturing the rigid cells in a buffer and depositing the rigid cells on the surface of a gold chip optical sensor system, can include, for example:

growing the rigid cells for a time within the late logarithm phase;

collecting the rigid cells by centrifugation or filtration;

washing the rigid cells with a suitable buffer such as saline;

vortexing the rigid cell suspension;

collecting the rigid cells by centrifugation or filtration;

optionally repeating the collecting, and washing, one or more times, to remove high refractive index materials, such as glucose, cell waste, and like materials;

counting the number of rigid cell; and

dispersing at least one dose of the counted rigid cells in a buffer on the optical sensor surface.

In embodiments, the density of cultured rigid cells in a buffer can be, for example, from about 100 to about 1,000,000 cells/mL.

In embodiments, the density or dose of cells deposited on the sensor can be, for example, from about 1,000 to about 100,000, such as from about 2,000 to about 50,000, from about 3,000 to about 25,000, from about 3,000 to about 5,000, including intermediate values and ranges. In a significant aspect, the dispersing of at least one dose of the counted cells in a buffer on the optical sensor surface can be, for example, about 3000 to about 5000 cells/mL, and more particularly about 4000 cells/mL per sensor well, such as yeast cells in saline.

In embodiments, the cells can be, for example, at least one of: yeast, fungi, algae, plant, coccus bacteria, clusters thereof, and like cells, or a combination thereof.

In embodiments, the gold chip optical sensor system can be, for example, a depth resolved cell assay system.

In embodiments, the two or more wavelengths can comprise at least two or more wavelengths of from about 380 nm to about 1,550 nm. The two or more penetration depths can be, for example, from about 3 to about 20 different depths, and the two or more penetration depths can be, for example, from about 30 to about 1500 nanometers.

In embodiments, the assay can provide an indication related to, for example, at least one of: assessment of a chemical effect on a rigid cell, drug screening on a rigid cell, diagnosis on a rigid cell, or a combination thereof.

In embodiments, the rigid cells can be, for example, substantially spheroidal cells. The substantially spheroidal cells can be, for example, spherical or elliptical, that is, oval- or egged-shaped cells having, for example, an aspect ratio of from about 1:1 to about 1:4.

In embodiments, the gold chip optical sensor system can be, for example, a Depth Resolved Cell Assay system, i.e., a DRCA™ system.

In embodiments, the disclosure provides a method for depth resolved sensing of a substantially spherical cellular entity, comprising:

irradiating the sensor of a surface plasmon resonance (SPR) sensor system having the rigid cellular entity on the sensor surface with at least two different wavelengths, each of the different wavelengths having a different penetration depth;

monitoring each of the different wavelengths for a change in the index of refraction; and

correlating the change in the index of refraction to a change in the biological entity.

In embodiments, the metal surface layer of the sensor chip can have, for example, an organic polymer, an inorganic entity or polymer, and like entities, or a combination thereof, applied as a coating thereto, prior to placing the rigid cell entity on the sensor chip surface.

In embodiments, the deposited rigid cells individually, or collectively, have only a very limited cell surface area, for example, from about 0.1 to about 5% total cell surface area, in contact with the sensor surface.

In embodiments, the assay including contacting the rigid cell with a substance can provide a useful tool, for example, in drug discovery, drug screening, medical diagnosis, medical treatment, cancer studies, and like utilities related to rigid cell related illness or conditions, or a combination thereof.

In embodiments, the rigid cells can be particles selected from, for example, a polymer, a glass, a composite, an inorganic material, a metal, a microballon, and like materials, or mixtures thereof.

Any aspect, feature, or embodiment of the foregoing disclosure can be used in any combination or permutation with any one or more other aspect, feature, or embodiment recited in the appended claims.

### EXAMPLES

The following examples serve to more fully describe the manner of using the above-described disclosure, and to further set forth the best modes contemplated for carrying out various aspects of the disclosure. It is understood that these examples do not limit the scope of this disclosure, but rather are presented for illustrative purposes. The working examples further describe how to make and use the disclosed methods.

### Materials

### Chemicals

Amphotericin, clotrimazole, fluconazole, and ketaconazole, and YPD medium were from Sigma (St. Louis, Mo.). Saccharomyces cerevisiae S288C yeast cells and phosphate saline buffer were from ATCC (Manassas, Va.). Yeast Peptone Dextrose (yeast growth media) (YPD) medium was from Invitrogen (Carlsbad, Calif.).

### Cell Culture

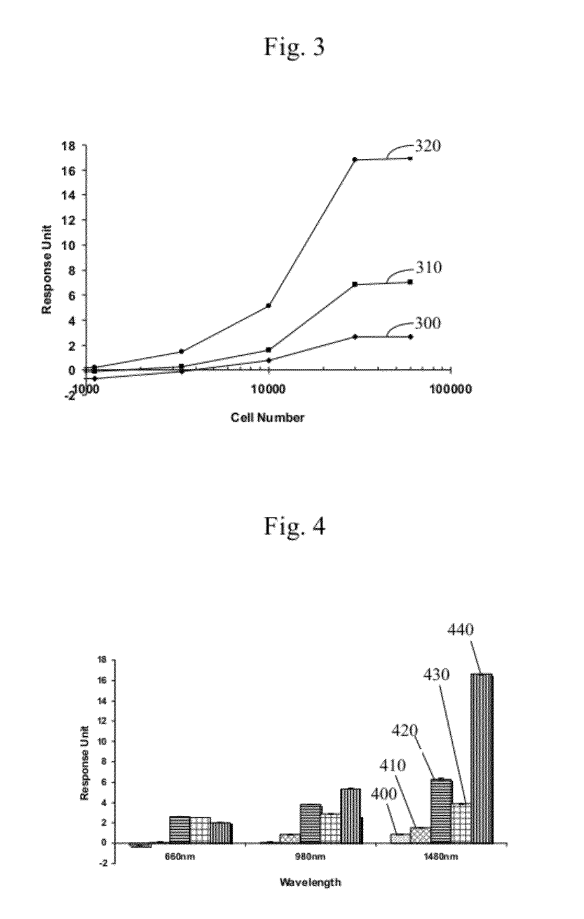

Yeast cells were cultured on an YPD agar plate at 28° C. for 16 hours and kept at 4° C. until just prior to use. From the agar plate, yeast cells were collected and seeded into YPD liquid medium and cultured at 28° C. for 8 hours. The cells were then diluted with its liquid medium to obtain a cell density of about 4,000 cells/mL as a starting point for cell inhibition studies. For the detection of yeast cells, the cells were collected at the late logarithm phase and collected by centrifugation at 10,000 g for 10 minutes. Cells were further washed to remove debris and/or high index material such as glucose. Yeast cells were added in different dose as indicated in the FIG. 3.

### Lab Apparatus

The UVO-cleaner was from Jelight Company Inc. (Laguna Hills, Calif.). The Vortex mixer was from VWR (Radnor, Pa.). The 8-channel electronic micro-channel pipette and hemocytometer were from Thermo Fisher Scientific (Hudson, N.H.). The spectrophotometer was from Agilent Technologies (Wilmington, Del.).

### DRCA System

A DRCA™ system equipped with three lasers having wavelengths of, for example, 660 nm, 980 nm, and 1480 nm, was selected for the study. The system allows for simultaneously measuring a cell at three different wavelengths. The output measures the angular change of the surface plasmon resonance (SPR). The angular change indicates a mass change within the defined sensing region. The depth of the sensing region depends on the wavelength of the selected illuminating laser as listed, for example, in Table 1.

TABLE 1
Correlation for wavelength and sensing depth.

| wavelength | sensing depth |
|---|---|
| (nm) | (nm) |
| 660 | 200 |
| 980 | 570 |
| 1480 | 1500 |

### Cell Assay

A user-assembled assay plate was first assembled with a gold coated glass slide and an 80-well holey plate (cut from a 384-well holey plate). Yeast cells were seeded into the wells with or without a compound and incubated in the instrument for approximately 60 minutes at 22° C. The cell responses were then measured and data were collected according to the description in the aforementioned U.S. Ser. No. 12/956,464.

A general procedure for accomplishing label-free yeast cell detection in accord with the present disclosure can include, for example:
1. turn on the DRCA optical system to warm-up the lasers, for example, for about 1 hour;
2. clean and assemble an 80-well holey plate on top of a gold chip;
3. sterilize the assembled chip by exposure to high intensity UV light for about 5 minutes;
4. grow yeast cells until the middle or late logarithm stage; in embodiments, the late logarithm stage is preferred to reduce yeast at budding status;
5. collect yeast cell by centrifugation, at e.g., 10,000 g for 10 minutes;
6. wash the cells with at least 5× fold volume of the pellet using a saline buffer, such as phosphate saline buffer;
7. vortex the cell solution using, for example, a bench top vortex mixer;
8. collect yeast cells by centrifugation at, e.g., 10,000 g for 10 minutes;
9. repeat steps 6 to 8 for approximately three (3) times to remove high refractive index materials, such as sugar residues, cell debris, or like materials, and mixtures thereof;
10. count the cell number using, e.g., a hemocytometer;
11. prepare different doses of yeast cells, such as, from 100 to 10,000 cells/mL, in a saline buffer, such as phosphate saline buffer;
12. prepare an assay buffer with or without a compound/bio-molecule;
13. load assay buffer in to each well of the cell chamber and equilibrate it, e.g., 60 minutes;
14. record the baselines, e.g., 5 minutes;
15. load a different dose of yeast cells;
16. monitor the response with a plurality of wavelengths and depths; and
17. record the data; and
18. analyze the data by calculating the difference between the end points and baselines.

In the foregoing general procedure, steps 4 to 11 differ from a mammalian cell assay procedure reported previously, see the aforementioned U.S. Ser. No. 12/956,464. and U.S. Ser. No. 12/947,066.

A procedure for accomplishing a label-free rigid cell assay response to a substance in accord with the present disclosure can include, for example:
1. clean and assemble an 80-well holey plate on top of a gold chip;
2. sterile the assembled chip by exposure to high intensity UV light for around 5 minutes;
3. grow yeast cells until the middle or late logarithm stage, preferably the former for an active inoculum;
4. turn on the DRCA optical system to warm-up the lasers for, e.g., 1 hour;
5. collect yeast cell by centrifugation at, e.g., 10,000 g for 10 minutes;
6. wash the cells with at least 5× fold volume of the pellet using a saline buffer, such as phosphate saline buffer;
7. vortex the cell solution using, for example, a bench top vortex mixer;
8. collect yeast cells by centrifugation at e.g. 10,000 g for 10 minutes;
9. repeat steps 6 to 8 for approximately three (3) times to remove high refractive index materials, such as, sugar residues, cell debris, or like materials, and mixtures thereof;
10. count yeast cell number using, e.g., a hemocytometer
11. prepare assay buffer with or without a substance (compound/bio-molecule);
12. load assay buffer with or without a substance (compound/bio-molecule) to each well of the cell chamber and let it equilibrate for, e.g., 60 minutes;
13. record the baselines for, e.g., 15 minutes;
14. add a small amount of yeast cells, such as 4000 cells/mL to each well;
15. monitor the yeast cell response, for example, to an added substance, with a plurality of wavelengths and depths;
16. record the data; and
17. analyze the data by calculating the difference between the end points and baselines.

In the foregoing assay procedure, steps 3 to 9 differ from a mammalian cell assay procedure reported previously, see the aforementioned U.S. Ser. No. 12/956,464, and U.S. Ser. No. 12/947,066.

### Example 1

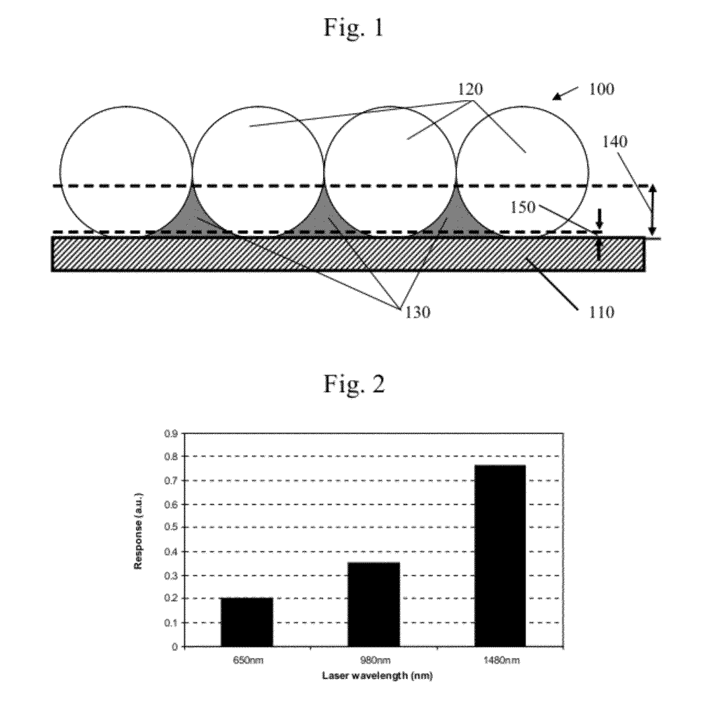

Modeling. To further understand the observed results, a yeast cell can be viewed as a uniform ball with a certain refractive index when seated in liquid. In this instance, any biological reaction with a substance can lead to a change in refractive index of the ball. When all the rigid target cells are sitting on the sensor chip surface, mixing with a liquid, the device senses the index change. In the modeling, the ball diameter is 5 micrometers and the liquid is water having refractive index of 1.33. The 5 micrometer thick rigid cel layer is divided into 30 sub-layers and each sub-layer has a different refractive index due to water/ball volume ratio. The SPR response is modeled by changing the refractive index of the ball from 1.35 to 1.37 while keeping water index refractive constant. The signal amplitude vs. laser wavelengths is plotted in FIG. 2. FIG. 2 shows that the short wavelength light produces the smallest signal due to its thinnest penetration depth in which the rigid cell (or the ball) is only a small portion of the whole sensing volume and results in only a small change. By contrast, the 1480 nm laser light shows the strongest signal change due to its deepest penetration depth. The modeling results agree very well with the actual rigid cell assay results.

Actual yeast cells differ from a uniform ball or sphere model since actual yeast cells have many biological elements with different indices. The yeast reaction with a substance (e.g., drug compounds) can be much more complex than just causing a uniform refractive index change. Nevertheless, the simplified model can be useful to differentiate responses from different laser wavelengths and to demonstrate the capabilities of disclosed method.

### Example 2

Detection of budding yeast cells. Yeast cells were serially diluted and added to each well of a well-plate. Their responsive units (RU) were measured with the DRCA™ system as a function of time (seconds). As seen in FIG. 3, there was a small signal (300) at the wavelength of 660 nm having about a 200 nm penetration depth. However, about a 3-fold increased signal (310) was observed with the 980 nm wavelength having a penetration depth of about 570 nm, and about an 11-fold increased signal (320) was observed with the 1480 nm wavelength having a penetration depth of about 1500 nm, relative to the signal observed at the wavelength of 660 nm. The results suggested that the penetration depth had a significant effect on assay sensitivity. The ability to measure a cell at multiple penetration depths allows for detecting rigid spherical cells that are not readily accessible with conventional methods, e.g., Epic® cell assay technology. The 660 nm, 980 nm, and 1480 nm are typical wavelengths used in the DRCA™ system.

### Example 3

Effect of modulators on yeast cells. Pathway modulators, including for example, a compound, a bio-molecule, or a mixture thereof, can be used, for example, to dissect cellular processes that can have a significant impact on cell biology study, and in drug discovery. Amphotericin is a polyene antifungal antibiotic from Streptomyces and has an affinity for sterols, primarily ergosterols, of fungal cell membranes, and forms channels in the membranes, causing small molecules to leak out. Ketaconazole is a first generation antifungal azole. Ketaconazole inhibits cytochrome P450-dependent 14α-demethylase, which is critical to ergosterol biosynthesis. The accumulated 14α-methylated sterols change the membrane structure of sensitive fungi, altering cell membrane permeability. Like ketaconazole, clotrimazole also inhibits cytochrome P450-dependent 14α-demethylase, and is highly selective. Fluconazole is a potent inhibitor of CYP2C9 and interferes with fungal ergosterol synthesis and down regulates the metallothionein gene.

Four modulator compounds were used to study of their effect on yeast cells. A user assembled (i.e., a virtual) assay plate was seeded with 4,000 cells per well. A modulator was added to a test well at 10 micromolar and then pre-incubated for 60 minutes. Each modulator was tested in three replicates. The well without a modulator was a positive control. The results, plotted in FIG. 4, showed that the compounds: amphotericin (Amph)(400), clotrimazole (Clot)(410), fluconazole (Fluc)(420), and ketaconazole (Keta)(430), (and no anti-yeast compound treatment was a positive control (PC, 440)), all had an effect on yeast cell growth at different levels. The effect of Fluc and Keta was not detectable when measuring at short penetration depth with the 660 nm laser source. Amphotericin and clotrimazole had a more noticeable effect on yeast cell growth than fluconazole and ketaconazole, which was consistent when measured at three different penetration depths. However, the difference between the modulators and controls was more substantial when measured at the longest penetration depth using the 1480 nm laser source. The data indicated that multiple wavelength detection provides greater information and insight into cellular biology compared to existing methods. Accordingly, the disclosed method can be useful for drug screening, drug development, and like applications.

### Example 4

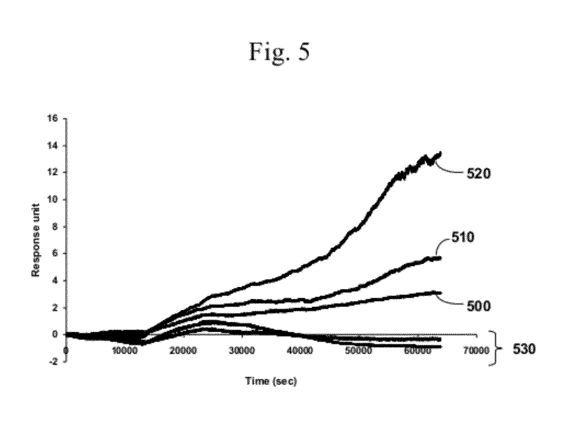

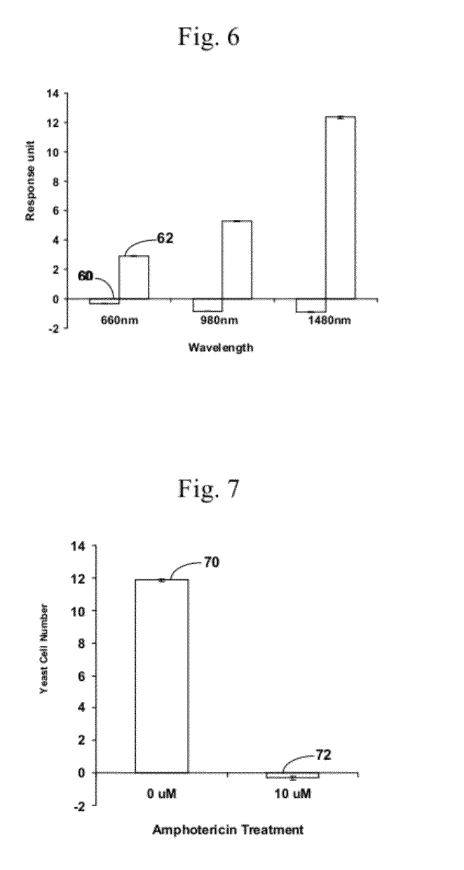

Time course of a modulator effect. The effect of an amphotericin compound on yeast cells was monitored in real time under ambient condition. As shown in FIG. 5, a user-assembled (virtual) assay plate was seeded with 4,000 cells per well. Amphotericin was added to a test well at 10 micromolar and pre-incubated for 60 minutes. The differential response was observed during the course of about 16 hours. With amphotericin treatment, there was almost no change in response measured at any given penetration depth as the compound could effectively inhibit yeast growth and produced the base line signal collectively labeled (530). As expected, the control without the amphotericin treatment showed increased response units as a function of time at 660 nm (500), 980 nm (510), and 1480 nm (520), indicating a normal cell growth pattern. The difference between the test well (500, 510, and 520) and the control well(s) (530) was increased as the measuring penetration depth was increased. The observed results support the concept that a longer penetration depth has a sensing advantage over shorter penetration depths (see also FIG. 6 amphotericin (60) and no amphotericin (62)) for several applications, such as the study of yeast cells, and measuring changes, for example, in the nuclear receptor.

FIG. 7 charts absorbance readings of a conventional turbidometric determination using a visible light spectrophotometer, for cultured yeast cells without amphotericin (left; amphotericin at 0 micromolar) and contacted with amphotericin (right; amphotericin at 10 micromolar). Turbidity can be used as an indirect measure of cell growth. Using a standard curve generated with serial dilutions of known number cells, one can estimate cell density in a turbid cell culture. The turbidity method showed that the decreased optical signal was correlated to the decreased turbidity of the cell culture, demonstrating that cell growth was inhibited by amphotericin and corroborating the SPR sensor determination.

### Example 5

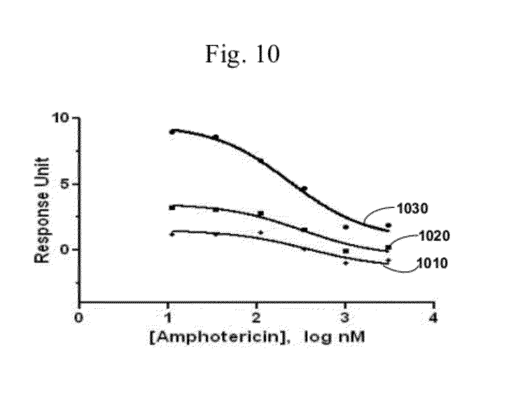

Dose-dependent Compound Effect. The inhibition curve of the amphotericin compound was further investigated. Amphotericin concentration at 3,000 nM was serially diluted at 3 fold. Yeast cells, about 4000 cells/well, were added for each treatment. While a typical inhibition curve was observed with all 3 wavelengths, the curve generated with the longest wavelength was more reliable (R²=0.9817 at 1480 nm>R²=0.9072 at 660 nm), see FIG. 10, where 660 nm (1010), IC₅₀=374 nM, R²=0.9072; 980 nm (1020), IC₅₀=271 nM, R²=0.9486; and 1480 nm (1030), IC₅₀=158 nM, R²=0.9817). The data indicated that the disclosed multiple depth assay provides considerably more biological related information compared to a single wavelength assay, such as more accurate determinations of, for example, substance efficacy, substance inhibition potency, substance cytotoxicity, and like substance information, or combinations thereof, with respect to substance impact on or interaction with the rigid cells.

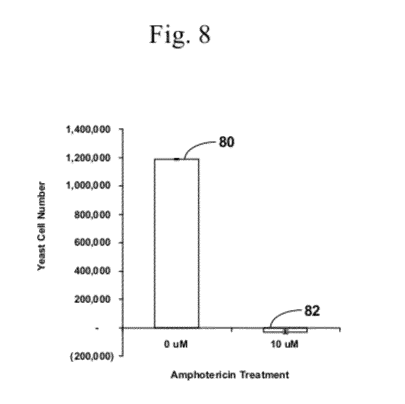

The samples from the aforementioned DRCA assay were further analyzed with a spectrophotometer (Agilent Technologies, Wilmington, Del.) and hemocytometer (Thermo Fisher Scientific, Hudson, N.H.). The absorbance reading from the sample that did not contain amphotericin had the OD580 of about 12, whereas the sample with 10 micromolar of amphotericin had OD580 of about zero (FIG. 7). Further, cell number counting results showed a similar trend. With amphotericin treatment, the cell number was about zero, compared to about 12,000,000 cells in the untreated sample, see FIG. 8 (yeast cell count by hemocytometer) for amphotericin (80) and no amphotericin (82) at each of three wavelengths. The data from the conventional assays further confirmed that the low DRCA signal is correlated with the low cell number that resulted from the growth inhibition by amphotericin, and demonstrates that the DRCA system could provide biologically meaningful data in the disclosed yeast assay.

FIG. 9 shows a typical logarithmic growth curve for yeast cells with respect to time. The early log phase (90), the mid log phase (92), the late log phase (94), and the stationary phase (96), are indicated with the corresponding reference numerals.

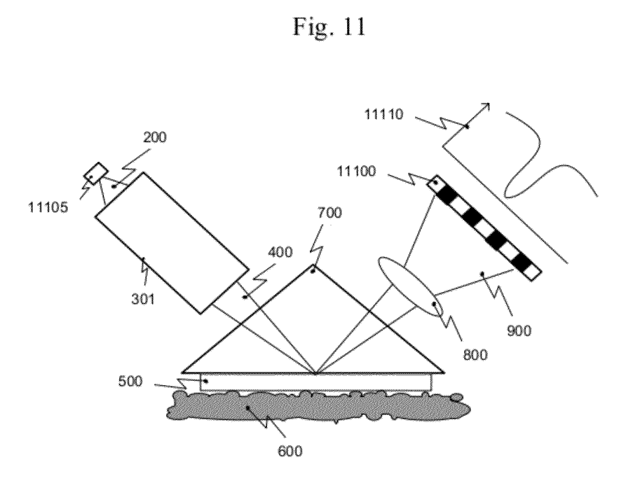

FIG. 11 shows an exemplary schematic of a multi-wavelength SPR sensing system using the sensor chip. FIG. 11 provides a general schematic of the disclosed SPR sensor system including a measurement apparatus having one or more light sources, several beam shaping optics, a high index of refraction coupling prism, a detection chip, one or more photodetector arrays, and a data acquisition unit. In embodiments, the SPR sensor system can include, for example, a light source (10); having at least two beams (20) from the light source; beam shaping optics (30); focused beams (40) for sensing; at least one sensor chip (50) or like chip(s); a user provided analyte or test specimen (60), such as a cell, a cell component, a cell construct, and like bio-entities; a prism (70); beam shaping optics (80); reflected or emitted beams (90); a photodetector array (100); and any suitable display, such as a graphical representation of the measured angular response of SPR (110).

A representative system having a single light source and a single photodetector array is illustrated. However, other variations can incorporate, for example, the addition of fiber or free-space couplers, fiber arrays, arrayed optics, beam splitters, or combination thereof, to enable a multiple source, multiple detector measurement, or both features into the apparatus. Although not limited by theory, the incident light excites surface plasmon resonance when the projection of the wave number of the incident light on the surface of the sensor matches that needed to generate the surface plasmon. The excitation of the surface plasmon wave and its subsequent dissipation on the metal film leads to the appearance of a minimum in the intensity versus angular distribution of the reflected light from the metal layer. The angular reflection minimum is extremely sensitive to the dielectric constant in the volume of the sample medium that is probed by the surface plasmon's evanescent field.

The light source of FIG. 11 can comprise, for example, one or more light emitting devices operating at different wavelengths that range from visible to near-IR wavelengths, for example, from about 400 nm to about 1,700 nm. The light sources can also have wavelengths across a large range (e.g., 400 to 1,700 nm).

The penetration depth of the evanescent field associated with SPR is a function of wavelength. The longer the wavelength, the deeper the surface plasmon can penetrate.

The sensor chip can comprise a transparent substrate having a metal sensing layer with a thickness of from about 30 nm to about 100 nm. The chip can have an optional adhesive layer between the substrate and the metal layer and can have a thickness of from about 1 nm to about 10 nm. Materials for the sensing layer can be gold, silver, copper, and like materials, or a combination thereof. The adhesive layer can be Ti, Ni, and like materials, or a combination thereof (see the aforementioned U.S. Ser. No. 12/956,464, and U.S. Ser. No. 12/947,066).

The disclosure has been described with reference to various specific embodiments and techniques. However, it should be understood that many variations and modifications are possible while remaining within the scope of the disclosure.

1. A label-free cell assay method comprising:
culturing rigid cells in a buffer, the rigid cells having a diameter of about 3 to about 7 micrometers;
depositing the rigid cells on the surface of a gold chip in a surface plasmon resonance optical sensor system; and
detecting the presence of the rigid cells on the sensor surface with the sensor system based on refraction changes when the deposited rigid cells are exposed to two or more wavelengths of light having two or more penetration depths.

2. The method of claim 1, further comprising:
contacting the deposited rigid cells with a substance; and
determining the differential response of the contacted rigid cells to the substance compared to rigid cells not contacted by the substance.

3. The method of claim 2, wherein determining the differential response of the rigid cells to the substance comprises at least one of:
measuring a baseline response of a buffer;
measuring a baseline response of the buffer and the rigid cells with the two or more wavelengths having two or more penetration depths;
measuring a response of the buffer and the substance contacted cells with the two or more wavelengths having two or more penetration depths; and
computing the difference between the measured response of the substance contacted cells and at least one baseline response.

4. The method of claim 1, wherein the sensor system comprises:
a light source providing at least two incident beams, each beam having at least one wavelength;
first optics providing incident beam shaping and beam focusing;
a sensor chip comprising a transparent substrate having on the first face of the substrate a high refractive index prism for receiving the incident focused beam, and having on the second face of the substrate having a metal layer of from about 30 nm to about 100 nm;
second optics providing reflected or emitted beam collection from the sensor;
a photodetector for receiving the collected beam and detecting the optical signal; and
a data acquisition unit, and the rigid cells are situated on the metal layer of the sensor chip surface.

5. The method of claim 1 wherein the rigid cells have a spherical or elliptical shape and have an aspect ratio of from about 1:1 to about 1:4.

6. The method of claim 5 wherein the rigid cells do not change their aspect ratio by more than about 1 to about 25%.

7. The method of claim 1, wherein the cultured rigid cells were collected at the logarithm growth phase prior to contacting.

8. The method of claim 7, wherein the logarithm phase comprises the time from the inflection point to the plateau of the growth curve.

9. The method of claim 1, wherein the deposited rigid cells were collected at the late logarithm stage of culture prior to contacting.

10. The method of claim 1, wherein culturing the rigid cells in a buffer and depositing the rigid cells on the surface of a gold chip optical sensor system comprises:
growing the rigid cells for a time within the late logarithm phase;
collecting the rigid cells by centrifugation or filtration;
washing the rigid cells with a saline buffer;
vortexing the cell suspension;
collecting the rigid cells by centrifugation or filtration;
optionally repeating the collecting and washing, one or more times, to remove high refractive index materials;
counting the number of rigid cells; and
dispersing at least one dose of the counted rigid cells in a buffer on the optical sensor surface.

11. The method of claim 1, wherein the density of rigid cells deposited on the sensor is from about 1,000 to about 100,000.

12. The method of claim 1 wherein the rigid cells comprise at least one of: yeast, fungi, algae, plant, bacteria, clusters thereof, or a combination thereof.

13. The method of claim 1 wherein the gold chip optical sensor system is a depth resolved cell assay system.

14. The method of claim 1 wherein the two or more beams comprise at least two or more wavelengths of from about 380 nm to about 1,550 nm.

15. The method of claim 1 wherein the two or more penetration depths comprise from about 3 to about 20 different depths, and the two or more penetration depths are from about 30 to about 1500 nanometers.

16. The method of claim 2 wherein the substance comprises at least one of: a molecule, biomolecule, a compound, an oligomer, a polymer, a modulator, an effector, a nutrient, a toxin, an inhibitor, a salt thereof, a gas, radiation, electricity, or combinations thereof.

17. The method of claim 2 wherein the deposited rigid cells individually have from about 0.1 to about 5% total surface area in contact with the sensor surface.

18. The method of claim 1 wherein the rigid cells can be particles selected from the group consisting of a polymer, a glass, a composite, an inorganic material, a metal, a microballon, and mixtures thereof.
